(19) United States
(12) Patent Application Publication
(10) Pub. No.: US 2015/0190060 A1
(43) Pub. Date: Jul. 9, 2015

(54) METHOD, DEVICE AND SYSTEM FOR CALCULATING INTEGRATED CAPNOGRAPH-OXIMETRY VALUES

(71) Applicant: ORIDION MEDICAL 1987 LTD., Jerusalem (IL)

(72) Inventors: Paul S. Addison, Midlothian (GB); James N. Watson, Fife (GB); James Ochs, Seattle, WA (US)

(73) Assignee: ORIDION MEDICAL 1987 LTD., Jerusalem (IL)

(21) Appl. No.: 14/148,682

(22) Filed: Jan. 6, 2014

(51) Int. Cl. A61B 5/0205; A61B 5/00; A61B 5/02; A61B 5/1455

(52) U.S. Cl. CPC A61B 5/0205; A61B 5/14551; A61B 5/726; A61B 5/7278; A61B 5/7282; A61B 5/7264; A61B 5/746; A61B 5/743; A61B 5/7275; A61B 5/02028; A61B 5/0836

(57) ABSTRACT

The present disclosure provides methods, devices and systems for calculating integrated breath status values comprising: defining breath cycles based on CO₂ waveforms; generating transformed signals based on PPG signals; calculating respiratory effort values based on the transformed PPG signals; and calculating integrated breath status values based on the defined breath cycles and the respiratory effort values. The disclosure also provides methods, devices and systems for calculating cardiovascular status values comprising: defining breath cycles based on a CO₂ waveforms; generating transformed signals based on a PPG signals; calculating pulse output values based on the transformed PPG signal; calculating pulse output per breath values based on the defined breath cycles and the calculated pulse output values; and determining integrated cardiovascular status values based on a comparison of a change in the pulse output per breath values and in the CO₂ waveforms.

### TECHNICAL FIELD

The present disclosure relates to methods, devices and systems for calculating integrated capnograph-oximetry values for use in identifying respiratory and cardiovascular conditions.

### BACKGROUND

Medical monitoring devices are routinely used in various medical settings to provide crucial data regarding a patient's medical condition.

Capnography is a non-invasive monitoring method used to continuously measure CO₂ concentration in exhaled breath. The exhaled CO₂, also known as end tidal CO₂ (EtCO₂), is an approximate estimation of the arterial levels of CO₂. The measurements of the CO₂ concentration in a breath cycle are performed by a capnograph, and the results are a numerical value displayed also in a graphical format in the shape of a waveform named a capnogram. The numerical value of the results may be presented in units of pressure (mm Hg) or a percentile. The capnogram may depict CO₂ concentration against total expired volume, but the more common capnogram illustrates CO₂ concentration against time.

An oximeter is a medical device that is used to determine the oxygen saturation of the blood. One common type of oximeter is a pulse oximeter, which may indirectly measure the oxygen saturation of a patient's blood and changes in blood volume in the skin. Ancillary to the blood oxygen saturation measurement, pulse oximeters may also be used to measure the pulse rate of the patient. Pulse oximeters typically measure and display various blood flow characteristics including, but not limited to, the oxygen saturation of hemoglobin in arterial blood.

### SUMMARY

The present disclosure relates to methods, devices and systems for calculating integrated breath status values comprising: defining individual breath cycles based on CO₂ waveforms; generating a transformed signal based on a photoplethysmograph (PPG) signal; calculating a respiratory effort value based on the transformed PPG signal; and calculating an integrated breath status value based on the defined breath cycle and the calculated respiratory effort value.

The present disclosure also relates to methods, devices and systems for calculating integrated cardiovascular status values comprising: defining individual breath cycles based on CO₂ waveforms; generating a transformed signal based on a PPG signal; calculating a pulse output value based on the transformed PPG signal; calculating a pulse output per breath value based on the defined breath cycle and the calculated pulse output value; and determining the integrated cardiovascular status value based on a comparison of a change in the pulse output per breath value and a change in the CO₂ waveform.

It is understood by the skilled in the art that the present disclosure provides numerous advantages. Inter alia, the combined capnograph-oximetry values such as the integrated breath status values and the integrated cardiovascular status values of the present disclosure may be of strong indicative and/or predicative value when assessing medical conditions.

For example, the present disclosure provides methods, devices and systems for assessing the severity of a respiratory condition, such as, but not limited to, asthma. According to some embodiments, assessing the severity of a respiratory condition includes identifying a critical and/or life threatening respiratory condition requiring immediate medical attention. Hence, based on the present disclosure, acute severe respiratory conditions, necessitating immediate medical attention, may be distinguished from moderate conditions, which may pass by without requiring medical interference.

Further to the above, the present disclosure also teaches triggering an alarm only if a severe acute condition is identified or alternatively triggering different alarms depending on the severity of the respiratory condition. As understood by those skilled in the art, the methods, devices and systems of the present disclosure may thus significantly reduce the number of false alarms and thereby avoid “cry wolf” situations where alarms are disregarded even when important.

In another example, the present disclosure provides methods, devices and systems for identifying cardiovascular conditions, such as, but not limited to, cardiac arrest. Advantageously, changes in pulse amplitude signals, such as those derived from a PPG sensor, may, according to the present disclosure, be used to determine whether a change in a CO₂ waveform is due to a cardiovascular irregularity/impediment. It is understood by the skilled in the art that rapid identification of cardiac arrest may be of uttermost importance in the attempt of saving lives.

In an alternative example, changes in a CO₂ waveform may be discarded as being due to a cardiovascular condition. Thus the methods, devices and systems of the present disclosure advantageously provide ways to differentiate between CO₂ waveform irregularities of different origin such as irregularities originating from cardiovascular conditions, respiratory conditions and other medical conditions.

Furthermore, the integrated cardiovascular status value may be used as a measure of the efficacy of resuscitation. As such, the integrated cardiovascular status value as described herein may be utilized to provide feedback to an emergency medical service (EMS) provider as to the quality of his resuscitation technique (e.g. CPR).

The following embodiments and aspects thereof are described and illustrated in conjunction with systems, tools and methods which are meant to be exemplary and illustrative, not limiting in scope. Certain embodiments of the present disclosure may include some, all, or none of the above advantages. One or more technical advantages may be readily apparent to those skilled in the art from the figures, descriptions and claims included herein. Moreover, while specific advantages have been enumerated above, various embodiments may include all, some or none of the enumerated advantages.

According to some embodiments, there is provided a method for calculating an integrated breath status value, the method comprising: defining a breath cycle based on a CO₂ waveform; generating a transformed signal based at least on a PPG signal; calculating a respiratory effort value based at least on the transformed PPG signal; and calculating an integrated breath status value based on the defined breath cycle and the calculated respiratory effort value.

According to some embodiments, the transformed PPG signal is a wavelet transformed signal. According to some embodiments, the wavelet transform is a continuous wavelet transform. According to some embodiments, generating the transformed signal comprises generating a scalogram. According to some embodiments, the integrated breath status value is an effort per breath value. Alternatively, the integrated breath status value is a weighted effort per breath value further based on at least one additional waveform related parameter, at least one additional PPG related parameter or combinations thereof. Non-limiting examples of additional waveform related parameter include: slope of the CO₂ waveform, slope in a plateau region of the waveform, amplitude of the waveform, angle of rise, time to rise, run time of the rise, curvature, acceleration, area under the waveform, baseline value of the waveform and any combination of these parameters. Each possibility is a separate embodiment. Non-limiting examples of additional PPG related parameter include: respiration rate, heart rate, blood pressure, saturation of peripheral oxygen (SpO2), fluid responsiveness, perfusion index (quantification of the amplitude of the peripheral plethysmograph waveform) and combinations thereof. Each possibility is a separate embodiment.

According to some embodiments, the method further comprises assessing the severity of a respiratory condition of a patient based on the integrated breath status value. Non-limiting examples of respiratory conditions include asthma and chronic obstructive pulmonary disease (COPD). According to some embodiments, assessing the severity of the respiratory condition comprises comparing the integrated breath status value to a predetermined threshold value. According to some embodiments, assessing the severity of the respiratory condition further comprises applying a fuzzy logic, a Bayesian network, a decision tree, a neural network, a radial base function, a linear regression model, a non-linear regression model, an expert system, or any combination thereof.

According to some embodiments, the method further comprises triggering an alarm when the integrated breath status value crosses the predetermined threshold value. According to some embodiments, the method further comprises identifying an improvement, deterioration or lack of change in the respiratory condition based on a trend in the integrated breath status value. According to some embodiments, the method further comprises displaying the integrated breath status value and/or the trend on one or more displays.

According to some embodiments, there is provided a device for calculating an integrated breath status value, the device comprising a processor configured to: define a breath cycle based on a CO₂ waveform; generate a transformed signal based at least on a PPG signal; calculate a respiratory effort value based at least on the transformed PPG signal; and calculate an integrated breath status value based on the defined breath cycle and the calculated respiratory effort value.

According to some embodiments, the processor is further configured to assess the severity of a respiratory condition by comparing the integrated breath status value to a predetermined threshold value. According to some embodiments, the processor is further configured to trigger an alarm when the integrated breath status value crosses the predetermined threshold value.

According to some embodiments, the processor is further configured to identify an improvement, deterioration or lack of change in the respiratory condition based on a trend in the integrated breath status value. According to some embodiments, the processor is further configured to display the integrated breath status value and/or the trend on one or more displays.

According to some embodiments, there is provided a system for calculating an integrated breath status value, the system comprising: a capnograph configured to provide CO₂ waveforms; a PPG sensor configured to provide PPG signals; and a processor configured to: define a breath cycle based on the CO₂ waveforms; generate a transformed signal based on the PPG signals; calculate a respiratory effort value based on the transformed PPG signal; and calculate an integrated breath status value based on the defined breath cycle and the calculated respiratory effort value.

According to some embodiments, the system further comprises an alarm configured to be triggered when the integrated breath status value crosses a predetermined threshold.

According to some embodiments, the system further comprises one or more displays configured to display the calculated integrated breath status value.

According to some embodiments, there is provided a method for calculating an integrated cardiovascular status value, the method comprising: defining a breath cycle based on a CO₂ waveform; generating a transformed signal based at least on a PPG signal; calculating a pulse output value based on the transformed PPG signal; calculating a pulse output per breath value based at least on the defined breath cycle and the calculated pulse output value; and determining the integrated cardiovascular status value based on a comparison of a change in the pulse output per breath value and a change in the CO₂ waveform.

According to some embodiments, the transformed PPG signal is a wavelet transformed signal. According to some embodiments, the wavelet transform is a continuous wavelet transform. According to some embodiments, generating the transformed signal comprises generating a scalogram. According to some embodiments, the change in the pulse output per breath value may proceed, follow or be concurrent with the change in the CO₂ waveform.

According to some embodiments, the method further comprises identifying a cardiovascular condition based on the integrated cardiovascular status value. Non-limiting examples of cardiovascular conditions include cardiac arrest, atrial flutter, atrial fibrillation, sinus tachycardia, ventricular tachycardia, ventricular fibrillation, bradycardia, bigeminy, trigeminy, ectopic beats, asystole and any combination thereof. Each possibility is a separate embodiment.

According to some embodiments, the method further comprises identifying an improvement, deterioration or lack of change in the cardiovascular condition. According to some embodiments, the method further comprises differentiating between cardiovascular conditions and respiratory conditions based on the integrated cardiovascular status value.

According to some embodiments, the method further comprises triggering an alarm when a cardiovascular condition is identified. According to some embodiments, the method further comprises displaying the integrated cardiovascular status value on one or more displays. According to some embodiments, the method further comprises calculating a confidence level of the integrated cardiovascular status value.

According to some embodiments, there is provided a device for calculating an integrated cardiovascular status value, the device comprising a processor configured to: define a breath cycle based on a CO₂ waveform; generate a transformed signal based at least on a PPG signal; calculate a pulse output value based on the transformed PPG signal; calculate a pulse output per breath value based at least on the defined breath cycle and the calculated pulse output value; and determine the integrated cardiovascular status value based on a comparison of a change in the pulse output per breath and a change in the CO₂ waveform.

According to some embodiments, the processor is further configured to identify a cardiovascular condition based on the integrated cardiovascular status value. According to some embodiments, the processor is further configured to identify an improvement, deterioration or lack of change in the cardiovascular condition. According to some embodiments, the processor is further configured to display the integrated cardiovascular status value on one or more displays. According to some embodiments, the processor is further configured to trigger an alarm when a cardiovascular condition is identified.

According to some embodiments, there is provided a system for calculating an integrated cardiovascular status value, the system comprising: a capnograph configured to provide CO₂ waveforms; a PPG sensor configured to provide PPG signals; and a processor configured to: define a breath cycle based on the CO₂ waveform; generate a transformed signal based at least on the PPG signal; calculate a pulse output value based on the transformed PPG signal; calculate a pulse output per breath value based at least on the defined breath cycle and the calculated pulse output value; and determine the integrated cardiovascular status value based on a comparison of a change in the pulse output per breath and a change in the CO₂ waveform.

According to some embodiments, the system further comprises an alarm configured to be triggered when a cardiovascular condition is identified. According to some embodiments, the system further comprises one or more displays configured to display the calculated integrated breath status value.

In addition to the exemplary aspects and embodiments described above further aspects and embodiments will become apparent by reference to the figures and by study of the following detailed description.

### BRIEF DESCRIPTION OF THE DRAWINGS

Examples illustrative of embodiments are described below with reference to figures attached hereto. In the figures, identical structures, elements or parts that appear in more than one figure are generally labeled with a same numeral in all the figures in which they appear. Alternatively, elements or parts that appear in more than one figure may be labeled with different numerals in the different figures in which they appear. Dimensions of components and features shown in the figures are generally chosen for convenience and clarity of presentation and are not necessarily shown in scale. The figures are listed below.

FIG. 1 is an illustrative flowchart depicting the steps used to determine an integrated breath status value in accordance with some embodiments.

FIG. 2 shows a perspective view of an exemplary system used to determine an integrated breath status value in accordance with some embodiments.

FIG. 3 is an illustrative flowchart depicting the steps used to determine an integrated cardiovascular status value in accordance with some embodiments.

FIG. 4 shows a perspective view of an exemplary system used to determine an integrated cardiovascular status value in accordance with some embodiments.

### DETAILED DESCRIPTION

In the following description, various aspects of the disclosure will be described. For the purpose of explanation, specific configurations and details are set forth in order to provide a thorough understanding of the different aspects of the disclosure. However, it will also be apparent to one skilled in the art that the disclosure may be practiced without specific details being presented herein. Furthermore, well-known features may be omitted or simplified in order not to obscure the disclosure.

The present disclosure relates to methods, devices and systems for calculating an integrated breath status value based on an integration of signals obtained from monitoring devices, such as, but not limited to, a capnograph and a PPG sensor. The present disclosure also relates to methods, devices and systems for calculating a cardiovascular status value based on an integration of signals obtained from monitoring devices, such as, but not limited to, a capnograph and a PPG sensor.

There is provided, in accordance with some embodiments, a method for calculating an integrated breath status value, the method comprising: defining a breath cycle based on a CO₂ waveform; generating a transformed signal based at least on a PPG signal; calculating a respiratory effort value based at least on the transformed PPG signal; and calculating an integrated breath status value based on the defined breath cycle and the calculated respiratory effort value.

It is understood by the skilled in the art that other biosignals, alternative or additional to the PPG signal can be implemented in the method and thus fall within the scope of this disclosure. Non-limiting examples of biosignals include: electrocardiogram, electroencephalogram, electrogastrogram, electromyogram, heart rate signals, pathological sounds, ultrasound, or any other suitable biosignal. Each possibility is a separate embodiment.

It is understood by the skilled in the art that other parameters, alternative or additional to CO₂ waveforms, may be utilized to determine a breath cycle and thus fall within the scope of this disclosure. Non-limiting examples include signals obtained from: a spirometer, a flow meter, an oximeter, an acoustic measurement device, non-dispersive infrared sensors, chemical gas sensors or any combination thereof. Each possibility is a separate embodiment.

It is understood by the skilled in the art, that the method of the disclosure is performed using a processer configured to execute the method as described.

As used herein the terms “breath status”, “respiratory status” and “ventilatory status” interchangeably relate to the medical state and/or condition of the airways, the lungs, and the respiratory muscles that mediate the movement of air into and out of the body.

As used herein the term “breath cycle” includes the stages of exhalation and inhalation. The breath cycle may be derived from a CO₂ waveform which depicts the change in expired CO₂ concentration over time, also known as end-tidal CO₂ (EtCO₂). During a breath cycle, the levels of CO₂ initially increase, as a result of CO₂ release from what is known as the “dead space”, which is the space in which no gas exchange takes place. Then, the CO₂ rapidly reaches a plateau at high levels of CO₂, which corresponds to the release of CO₂ from the lungs, in the exhalation phase. A rapid decline in exhaled CO₂ proceeds the inhalation phase, characterized by absence/minute levels of CO₂.

As used herein a “CO₂ waveform” and “capnogram” interchangeably refer to measurements of the CO₂ concentration over time, displayed in a graphical format. It is understood by the skilled in the art that a CO₂ waveform may refer to a single CO₂ waveform. Alternatively, the term may refer to a plurality of waveforms (for example 2, 3, 4, 5 or 10 waveforms), such as successive waveforms depicted in a capnogram. Each possibility is a separate embodiment.

As used herein the term “PPG signal” refers to the signal obtained from an oximeter such as for example a pulse oximeter configured to determine the oxygen saturation of the blood. The oximeter may pass light using a light source through blood perfused tissue and photoelectrically sense the absorption of light in the tissue. For example, the oximeter may measure the intensity of light that is received at the light sensor as a function of time. A signal representing light intensity versus time or a mathematical manipulation of this signal (e.g., a scaled version thereof, a log taken thereof, a scaled version of a log taken thereof, etc.) may be referred to as the PPG signal. In addition, the term “PPG signal,” as used herein, may also refer to an absorption signal (i.e., representing the amount of light absorbed by the tissue) or any suitable mathematical manipulation thereof. The light intensity or the amount of light absorbed may then be used to calculate the amount of the blood constituent (e.g., oxyhemoglobin) being measured as well as the pulse rate and when each individual pulse occurs. Pulse oximeters may also be used to determine respiratory effort in accordance with the present disclosure.

As used herein the terms “transformed biosignal”, “transformed PPG signal” and transformed signal” interchangeably refer to signals transformed using a continuous wavelet transform as described in the art.

Alternatively, the transformed signal may be a Fourier, spectral, scale, time, time-spectral, or time-scale transformed signal.

The continuous wavelet transform decomposes a signal using wavelets, which are generally highly localized in time. The continuous wavelet transform may provide a higher resolution relative to discrete transforms, thus providing the ability to garner more information from the signals. Continuous wavelet transforms allow for the use of a range of wavelets with scales spanning the scales of interest of a signal such that small scale signal components correlate well with the smaller scale wavelets and thus manifest at high energies at smaller scales in the transform. Likewise, large scale signal components correlate well with the larger scale wavelets and thus manifest at high energies at larger scales in the transform. Thus, components at different scales may be separated and extracted in the wavelet transform domain.

Wavelet transforms, such as continuous wavelet transforms, may be defined in a three-dimensional coordinate system and generate a surface with dimensions of time, scale and, for example, amplitude. As an alternative to amplitude, parameters such as but not limited to energy density, modulus and phase, may all be generated using such transforms.

The continuous wavelet transform of a signal x(t) in accordance with the present disclosure may be defined as:

$$T ( a , b ) = \frac{1}{\sqrt{a}} {\int }_{- ∞}^{+ ∞} x ( t ) ψ * ( \frac{t - b}{a} ) d t \qquad ( 1 )$$

According to some embodiments, any suitable wavelet function may be used in connection with the present disclosure. Commonly used complex wavelets include the Morlet wavelet.

According to some embodiments, calculating an integrated breath status value, further comprises generating a scalogram based on the wavelet transformed PPG parameter.

As used herein the term “scalogram” refers to a visual method of displaying a wavelet transform.

The scalogram, in accordance with the present disclosure may be defined as:

S(a,b)=|T(a,b)|² (2)

where ‘| |’ is the modulus operator. The scalogram may be resealed and used for defining ridges in wavelet space when, for example, the Morlet wavelet is used. Ridges are defined as the locus of points of local maxima in the plane. Any reasonable definition of a ridge may be employed in the method. Also included as a definition of a ridge herein are paths displaced from the locus of the local maxima. A ridge associated with only the locus of points of local maxima in the plane is labeled a “maxima ridge”.

In the discussion of the technology which follows herein, the “scalogram” may be taken to include all suitable forms of resealing including, but not limited to, the original unscaled wavelet representation, linear resealing, any power of the modulus of the wavelet transform, or any other suitable resealing. In addition, for purposes of clarity and conciseness, the term “scalogram” shall be taken to mean the wavelet transform, T(a,b) itself, or any part thereof.

As used herein the terms “effort”, “effort value”, breathing effort” and “respiratory effort” interchangeably refer to physical effort or work of a process such as for example effort of breathing. The respiratory effort may in turn affect respiratory signals, such as, but not limited to, a PPG signal. Respiratory effort may increase, for example, if a patient's respiratory pathway becomes restricted or blocked. Conversely, respiratory effort may decrease as a patient's respiratory pathway becomes unrestricted or unblocked.

The act of breathing may cause a breathing band to become present in a scalogram derived from a PPG signal. This band may occur at or about the scale having a characteristic frequency that corresponds to the breathing frequency. Changes in the respiratory effort of a patient may induce or change various features of the signal used to generate the scalogram. Therefore, the features within this band may be correlated with the patient's breathing effort.

According to some embodiments, the integrated breath status value is an effort per breath value. According to some embodiments, the term “per breath” refers to effort values measured during single breaths. According to some embodiments, the term “per breath” refers to effort values from which changes due to the respiratory cycle are discounted.

According to some embodiments, the integrated breath status value is an weighted effort per breath value further based on at least one additional waveform related parameter, at least one additional PPG related parameter or combinations thereof.

For example, the effort per breath value may receive an increased weight if the respiration rate exceeds a predetermined value. For example, the effort per breath value may receive an increased weight if the heart rate exceeds a predetermined value.

According to some embodiments, the at least one additional waveform related parameter is selected from the group consisting of: slope of the CO₂ waveform, slope in a plateau region of the waveform, amplitude of the waveform, angle of rise, time to rise, run time of the rise, curvature, acceleration, area under the waveform, baseline value of the waveform and any combination of these parameters. Each possibility is a separate embodiment.

According to some embodiments, the at least one additional PPG related parameter is selected from the group consisting of: respiration rate, heart rate, blood pressure, saturation of peripheral oxygen (SpO2), fluid responsiveness, perfusion index and combinations thereof. Each possibility is a separate embodiment.

According to some embodiments, the method for calculating an integrated breath status value further comprises assessing the severity of a respiratory condition of a patient based on the integrated breath status value. Non-limiting examples of respiratory conditions includes asthma, chronic obstructive pulmonary disease (COPD), lung injury, pneumonia and combinations thereof. Each possibility is a separate embodiment.

According to some embodiments, assessing the severity of a respiratory condition includes identifying a critical and/or life threatening respiratory condition requiring immediate medical attention.

According to some embodiments assessing the severity of a respiratory condition includes combining and/or integrating changes in CO₂ waveform related parameters and PPG related parameters such as SpO₂ values.

Changes in the shape of a CO₂ waveform may be indicative of a number of medical conditions, for example respiratory problems (such as asthma). During an asthma attack, the patient struggles to exhale, and as a result thereof, the increase in exhaled CO₂ is impeded. In addition the value of CO₂ will change, typically by first dropping (e.g. below 35 mmHg), then rising to normal, before rising to dangerously high levels (e.g. 60 mmHg), as the patient tires and has little air movement. During such asthma attack, the level of SpO₂ may remain fairly constant until the attack worsens and the effort signal in the patient baseline increases as the patient struggles to exhale, followed by a decrease when the patient tires. Hence, according to some embodiments, the effort signal derived from the PPG signal may be used to indicate attack severity.

According to some embodiments, a critical and/or life threatening respiratory condition is identified when the CO₂ value exceeds/crosses a predetermined threshold and when the effort of breathing goes below/crosses a predetermined threshold. According to some embodiments, assessing the severity of the respiratory condition comprises comparing the integrated breath status value to a predetermined threshold value. The threshold values may be based at least in part on empirical data, baseline readings, average readings, or a combination of data.

According to some embodiments, assessing the severity of the respiratory condition further comprises applying a fuzzy logic, a Bayesian network, a decision tree, a neural network, a radial base function, a linear regression model, a non-linear regression model, an expert system, or any combination thereof.

According to some embodiments, assessing the severity of the respiratory condition further comprises applying machine learning techniques such as cost function minimization, genetic programming, inductive logic programming as well as other techniques known in the art. For example, previously collected data, from patients with known respiratory conditions, may be used to teach a genetic programming system through minimizing a cost function describing the proportion of wrongly judged patient severity in this teaching set.

According to some embodiment, a severe/life threatening condition (such as for example an acute severe asthma attack) may be identified when the weighted effort per breath value exceeds a predetermined threshold. According to some embodiment, a severe/life threatening condition (such as for example an acute severe asthma attack) may be identified when the effort per breath value exceeds a predetermined threshold and when the least one additional waveform related parameter or the at least one additional PPG parameter exceeds a predetermined value. As a non-limiting illustrative example, a severe/life threatening condition may be identified when the integrated breath status value crosses a predetermined threshold and when a drop in blood pressure is observed.

According to some embodiments, the method further comprises identifying an improvement, deterioration or lack of change in the respiratory condition based on a trend in the integrated breath status value. According to some embodiments a “trend” as used herein may refer to values such as standard deviations, variance, dispersion, slopes, and the like, of the data or any other measured or calculated parameters that may indicate consistency and stable trends, or oppositely, indicate inconsistency

According to some embodiments, the method further comprises displaying the integrated breath status value and/or the trend on a display. According to some embodiments, displaying the integrated breath status value and/or the trend comprises color-coding the CO₂ waveform based on the respiratory effort derived from the transformed PPG signal. For example, low effort breaths may be depicted as a green CO₂ waveform. Alternatively, low effort breaths may be depicted as filed in green under the CO₂ waveform curve. For example, high effort breaths may be depicted as a red CO₂ waveform. Alternatively, high effort breaths may be depicted as filled in red under the CO₂ waveform curve. It is understood by the skilled in the art that additional and/or alternative method of displaying the integrated breath status value and/or the trend can be utilized and as such are encompassed by the scope of the disclosure.

According to some embodiments, the method further comprises triggering an alarm when the integrated breath status value and/or trend cross the predetermined threshold value. According to some embodiments the alarm triggered may differ according to the deviation of the integrated breath status value and/or trend from the predetermined threshold value. According to some embodiments the alarm triggered may differ according to the severity of the respiratory condition.

There is provided, in accordance with some embodiments, a device for calculating an integrated breath status value, the device comprising a processor configured to: define a breath cycle based on a CO₂ waveform; generate a transformed signal based at least on a PPG signal; calculate a respiratory effort value based at least on the transformed PPG signal; and calculate an integrated breath status value based on the defined breath cycle and the respiratory effort value.

It is understood by the skilled in the art that the processor is configured to implement the method described hereinabove.

According to some embodiments, the processor is further configured to assess the severity of a respiratory condition by comparing the integrated breath status value to a predetermined threshold value as essentially described above.

According to some embodiments, the processor is further configured to identify an improvement, deterioration or lack of change in the respiratory condition based on a trend in the integrated breath status value.

According to some embodiments, the processor is further configured to trigger an alarm when the integrated breath status value crosses the predetermined threshold value. According to some embodiments, the processor is further configured to trigger an alarm when a deterioration in the respiratory condition is identified. According to some embodiments, the processor is further configured to trigger an alarm when a deterioration or a lack of change in the respiratory condition is identified According to some embodiments, the processor is further configured to display the integrated breath status value and/or the trend on a display.

There is provided, in accordance with some embodiments, a system for calculating an integrated breath status value, the system comprising: a capnograph configured to provide CO₂ waveforms; a PPG sensor configured to provide PPG signals; and a processor configured to: define a breath cycle based on the CO₂ waveforms; generate a transformed signal based on the PPG signals; calculate a respiratory effort value based on the transformed PPG signal; and calculate an integrated breath status value based on the defined breath cycle and the calculated respiratory effort value.

It is understood by the skilled in the art that other biosensors, alternative or additional to the PPG signal can be incorporated into the system. Non-limiting examples of biosensors include: electrocardiograph, electroencephalograph, electrogastrograph, electromyograph, heart rate sensor, pathological sounds sensor, ultrasound sensor, or any other suitable biosensor. Each possibility is a separate embodiment.

It is understood by the skilled in the art that other sensors, facilitating identification of a breath cycle, may be implemented by the method and thus fall within the scope of this disclosure. Non-limiting examples of suitable sensors include a spirometer, a flow meter, an oximeter, an acoustic measurement device, non-dispersive infrared sensors, chemical gas sensors or any combination thereof. Each possibility is a separate embodiment.

According to some embodiments, the system further comprises an alarm configured to be triggered when the integrated breath status value crosses a predetermined threshold.

According to some embodiments, the system further comprises a display configured to display the calculated integrated breath status value.

There is provided, in accordance with some embodiments, a method for calculating an integrated cardiovascular status value, the method comprising: defining individual breath cycles based on CO₂ waveforms; generating a transformed signal based at least on a PPG signal; calculating a pulse output value based on the transformed PPG signal; calculating a pulse output per breath value based at least on the defined breath cycle and the calculated pulse output value; and determining the integrated cardiovascular status value based on a comparison of a change in the pulse output per breath value and a change in the CO₂ waveform.

It is understood by the skilled in the art that other biosignals, alternative or additional to the PPG signal can be implemented in the method. Non-limiting examples of such biosignals include: electrocardiogram, electroencephalogram, electrogastrogram, electromyogram, heart rate signals, pathological sounds, ultrasound, or any other suitable biosignal. Each possibility is a separate embodiment.

As used herein the term “pulse output value” refers to a pulse component derived from a PPG signal. For example, the PPG signal may be analyzed to identify/calculate the length, amplitude, or both of a single downstroke portion of a pulse and an average value (e.g., mean, median) of the length, amplitude, or both of the downstroke portions of pulses over a selected amount of time or over a selected number of cardiac cycles. In other examples, the PPG signal may be analyzed to identify/calculate the period of a single pulse and an average value (e.g., mean, median) of the period of pulses over a selected amount of time or over a selected number of cardiac cycles. In another example, the PPG signal may be analyzed to identify/calculate the area of a single pulse and an average value (e.g., mean, median) of the area of pulses over a selected amount of time or over a selected number of cardiac cycles. In another example, the PPG signal may be used to identify a pulse band within the PPG signal. As used herein, the term “pulse band” refers to the pulse component of a PPG signal which may produce a dominant band in wavelet space at or around the pulse frequency.

As used herein the term “breath cycle” refers to a breath which includes the stages of exhalation and inhalation. The breath cycle may be derived from a CO₂ waveform which depicts the change in expired CO₂ concentration over time, also known as end-tidal CO₂ (EtCO₂). During a breath cycle, the levels of CO₂ initially increase, as a result of CO₂ release from what is known as the “dead space”, which is the space in which no gas exchange takes place. Then, the CO₂ rapidly reaches a plateau at high levels of CO₂, which corresponds to the release of CO₂ from the lungs, in the exhalation phase. A rapid decline in exhaled CO₂ proceeds the inhalation phase, characterized by absence/minute levels of CO₂.

As used herein a “CO₂ waveform” and “capnogram” interchangeably refer to measurements of the CO₂ concentration over time, displayed in a graphical format. It is understood by the skilled in the art that a CO₂ waveform may refer to a single CO₂ waveform. Alternatively, the term may refer to a plurality of waveforms (for example 2, 3, 4, 5 or 10 waveforms), such as successive waveforms depicted in a capnogram. Each possibility is a separate embodiment.

As used herein the term “per breath” refers to pulse output values measured during single breaths. According to some embodiments, the term “per breath” refers to pulse output values from which changes due to the respiratory cycle are discounted.

As used herein a “change in the “CO₂ waveform” refer to a change in any of the “CO₂ parameters which may be extracted from the waveform, such as, but not limited to, the slope of the CO₂ concentration curve, the slope in a plateau region of the waveform, the amplitude of the waveform, the angle of rise, the time to rise, the run time of the rise, the curvature, the acceleration, the area under the curve, the baseline value and any combination of these parameters. Each possibility is a separate embodiment.

As used herein the terms “transformed biosignal”, “transformed PPG signal” and transformed signal” interchangeably refer to signals transformed using a continuous wavelet transform as described in the art.

Alternatively, the transformed signal may be a Fourier, spectral, scale, time, time-spectral, or time-scale transformed signal.

The continuous wavelet transform decomposes a signal using wavelets, which are generally highly localized in time. The continuous wavelet transform may provide a higher resolution relative to discrete transforms, thus providing the ability to garner more information from the signals. Continuous wavelet transforms allow for the use of a range of wavelets with scales spanning the scales of interest of a signal such that small scale signal components correlate well with the smaller scale wavelets and thus manifest at high energies at smaller scales in the transform. Likewise, large scale signal components correlate well with the larger scale wavelets and thus manifest at high energies at larger scales in the transform. Thus, components at different scales may be separated and extracted in the wavelet transform domain.

Wavelet transforms, such as continuous wavelet transforms, may be defined in a three-dimensional coordinate system and generate a surface with dimensions of time, scale and, for example, amplitude. As an alternative to amplitude, parameters such as but not limited to energy density, modulus and phase, may all be generated using such transforms.

The continuous wavelet transform of a signal x(t) in accordance with the present disclosure may be defined as:

$$T ( a , b ) = \frac{1}{\sqrt{a}} {\int }_{- ∞}^{+ ∞} x ( t ) ψ * ( \frac{t - b}{a} ) d t \qquad ( 1 )$$

According to some embodiments, any suitable wavelet function may be used in connection with the present disclosure. Commonly used complex wavelets include the Morlet wavelet.

According to some embodiments, calculating an integrated cardiovascular status value, further comprises generating a scalogram based on the wavelet transformed PPG parameter.

As used herein the term “scalogram” refers to a visual method of displaying a wavelet transform.

The scalogram, is defined as:

S(a,b)=|T(a,b)|² (2)

where ‘| |’ is the modulus operator. The scalogram may be resealed and used for defining ridges in wavelet space when, for example, the Morlet wavelet is used. Ridges are defined as the locus of points of local maxima in the plane. Any reasonable definition of a ridge may be employed in the method. Also included as a definition of a ridge herein are paths displaced from the locus of the local maxima. A ridge associated with only the locus of points of local maxima in the plane is labeled a “maxima ridge”.

In the discussion of the technology which follows herein, the “scalogram” may be taken to include all suitable forms of resealing including, but not limited to, the original unscaled wavelet representation, linear resealing, any power of the modulus of the wavelet transform, or any other suitable resealing. In addition, for purposes of clarity and conciseness, the term “scalogram” shall be taken to mean the wavelet transform, T(a,b) itself, or any part thereof.

As used herein the terms “cardiovascular condition”, “cardiovascular abnormality” and “cardiovascular irregularity” relate to the medical state and/or condition of the cardiovascular system. It is understood by the skilled in the art that changes in the medical state and/or cardiovascular condition may in turn affect both direct and indirect cardiovascular measurements. For example, changes in the medical state and/or cardiovascular condition may affect PPG signals and CO₂ waveforms.

It is well known by the skilled in the art that cardiovascular conditions may lead to a decreased blood flow and thus to a decreased presentation of CO₂ to the lungs. In effect a decrease in end-tidal CO₂ concentrations is evident in CO₂ measurements such as capnography.

Likewise, cardiovascular conditions may lead to a changes in the pulse amplitude and hence to changes in PPG signal morphology, such as, but not limited to, amplitude, timing, period, morphology, or other characteristics of the PPG signal. The pulse component of a PPG signal may produce a dominant band in wavelet space at or around the pulse frequency. By employing a suitable resealing of the scalogram, such as that given in equation (2), a pulse output value, such as for example a pulse rate, may be obtained from the PPG signal. Furthermore, by generating the wavelet transform, individual pulses may be captured. In this way, both times between individual pulses and the timing of components within each pulse may be monitored and used to detect heart beat anomalies, measure arterial system compliance, or perform any other suitable calculations or diagnostics.

For example, irregular pulses may be detected by comparing the length or the amplitude of a downstroke to a mean or median value. When the length of the amplitude of an individual downstroke exceeds a threshold value (e.g., 1.8 times the median value, or any other predetermined or calculated value), the downstroke may be identified as corresponding to an abnormal pulse. In another example, abnormal pulses may be detected by comparing the period of a pulse against a mean or median value. When the period of an individual pulse exceeds a threshold value (e.g., 1.8 times the median value), the pulse may be identified as corresponding to an abnormal pulse. Abnormal pulses may also be detected by comparing the area of a pulse against a running mean or median value.

According to some embodiments, capnography and photoplethysmography may be combined and/or integrated for example in order to establish whether a change in a CO₂ waveform is due to a change in a cardiovascular condition. A change in the pulse output value (such as for example an increase in pulse amplitude over time) accompanied by an increase in exhaled CO₂ may for example provide a firm indication that the increased CO₂ is due to a change in a cardiovascular condition, whereas an increase in CO₂ without a reliably correlated change in the pulse output value may discount a change in CO₂ as a potential change in a cardiovascular condition.

For example, during controlled ventilation, the absence of a pulse output value, such as in the absence of a pulse component, together with a rapidly decreasing capnogram waveform amplitude may indicate the presence of a cardiac arrest. For example, for free breathing patients, the absence of a pulse output value, such as for example in the absence of a pulse component, together with cessation of measured breathing may be indicative of cardiac arrest.

Non-limiting examples of cardiovascular conditions which may be identified by the method of the present disclosure include: cardiac arrest, atrial flutter, atrial fibrillation, sinus tachycardia, ventricular tachycardia, ventricular fibrillation, bradycardia, bigeminy, trigeminy, isolated irregularities such as ectopic beat, asystole and combinations thereof. Each possibility is a separate embodiment.

According to some embodiments, the method further enables identifying improvements, deteriorations or lack of change in the cardiovascular condition. For example, regaining a pulse output value and restoration of the CO₂ waveform may indicate an improvement in the cardiovascular condition of the patient such but not limited to successful resuscitation of the patient.

According to some embodiments, the change in the pulse output per breath value may proceed, follow or be concurrent with the change in the CO₂ waveform. For example a change in the pulse output per breath value (such as for example cardiac pulse arrhythmia) may be identified prior to a cardiac arrest and hence prior to the change in the CO₂ waveform (such as for example a decrease in or absence of an area under the curve due to a rapidly decreasing waveform or due to cessation of measured breathing). It is understood by the skilled in the art that knowledge of a recent history of cardiac pulse arrhythmia may assist in rapidly identifying a cardiovascular condition. For example, a recent history of cardiac pulse arrhythmia may enable to momentarily identify cardiac arrest when a pulse is subsequently lost and the patient stops breathing. According to some embodiment, knowledge of a recent history of cardiac pulse arrhythmia may facilitate the anticipation of cardiac arrest and thereby provide lifesaving medical attention prior to the attack.

According to some embodiments, the method further comprises displaying the integrated cardiovascular status value on a display. According to some embodiments, displaying the integrated cardiovascular status value comprises color-coding the CO₂ waveform based on the pulse output value. For example, abnormal pulse output values may be incorporated into the capnogram by coloring the curve in red. Alternatively, abnormal pulse output values may be depicted as a red filling of the area under the CO₂ waveform curve. It is understood by the skilled in the art that additional and/or alternative method of displaying the integrated breath status value and/or the trend can be utilized and as such are encompassed by the scope of the disclosure.

According to some embodiments, the method further comprises triggering an alarm when a cardiovascular condition is identified. Additionally or alternatively, the method further comprises triggering an alarm when the integrated cardiovascular status value crosses a predetermined threshold value. According to some embodiments the alarm triggered may differ according to the deviation of the integrated cardiovascular status value from the predetermined threshold value. According to some embodiments the alarm triggered may differ according to the severity of the cardiovascular condition.

According to some embodiments, the method further comprises determining a confidence value for the integrated cardiovascular status value. For example a representative integrated cardiovascular status value may be identified by combining multiple integrated cardiovascular status values (for example by calculating a mean or median value) and the standard deviation/variation may be used as an indication of stability of the measurements and hence provide a basis of confidence in the reading. Additionally or alternatively, the integrated cardiovascular status value may be weighted according to the degree of correlation between the pulse output per breath value and the CO₂ waveform. For example periods of strong correlation between the pulse output per breath value and the CO₂ waveform may receive a higher confidence value than periods of weak or negative correlation.

According to some embodiments, identifying a cardiovascular condition further comprises applying a fuzzy logic, a Bayesian network, a decision tree, a neural network, a radial base function, a linear regression model, a non-linear regression model, an expert system, or any combination thereof.

According to some embodiments, identifying a cardiovascular condition further comprises applying machine learning techniques such as cost function minimization, genetic programming, inductive logic programming as well as other techniques known in the art.

It is understood by the skilled in the art, that the method of the disclosure may be performed using a processer configured to execute the method as described.

There is provided, in accordance with some embodiments, a device for calculating an integrated cardiovascular status value, the device comprising a processor configured to: define individual breath cycles based on obtained CO₂ waveforms; generate a transformed signal based at least on a PPG signal; calculate a pulse output value based on the transformed PPG signal; calculate a pulse output per breath value based at least on the defined breath cycle and the calculated pulse output value; and determine the integrated cardiovascular status value based on a comparison of a change in the pulse output per breath and a change in the CO₂ waveform.

According to some embodiments, the processor is further configured to identify a cardiovascular condition based on the integrated cardiovascular status value. According to some embodiments, the processor is further configured to identify an improvement, deterioration or lack of change in the cardiovascular condition.

According to some embodiments, the processor is further configured to trigger an alarm when a cardiovascular condition is identified. According to some embodiments, the processor is further configured to display the integrated cardiovascular status value on a display.

It is understood by the skilled in the art that the processor is configured implement the method as essentially described above.

There is provided, in accordance with some embodiments, a system for calculating an integrated breath status value, the system comprising: a capnograph configured to provide CO₂ waveforms; a PPG sensor configured to provide PPG signals; and a processor configured to: define individual breath cycles based on the CO₂ waveforms; generate a transformed signal based at least on the PPG signal; calculate a pulse output value based on the transformed PPG signal; calculate a pulse output per breath value based at least on the defined breath cycle and the calculated pulse output value; and determine the integrated cardiovascular status value based on a comparison of a change in the pulse output per breath and a change in the CO₂ waveform.

It is understood by the skilled in the art that other biosensors, additional or alternative to the PPG sensor can be incorporated into the system. Non-limiting examples of biosensors include: electrocardiograph, electroencephalograph, electrogastrograph, electromyograph, heart rate sensor, pathological sounds monitor, ultrasound monitor, or any other suitable biosensor. Each possibility is a separate embodiment.

It is understood by the skilled in the art that other sensors, facilitating identification of a breath cycle, may be implemented by the method and thus fall within the scope of this disclosure. Non-limiting examples of suitable sensors include a spirometer, a flow meter, an oximeter, an acoustic measurement device, non-dispersive infrared sensors, chemical gas sensors or any combination thereof. Each possibility is a separate embodiment.

According to some embodiments, the system further comprises an alarm configured to be triggered when a cardiovascular condition/abnormality is identified. According to some embodiments, the system further comprises a display configured to display the integrated cardiovascular status value.

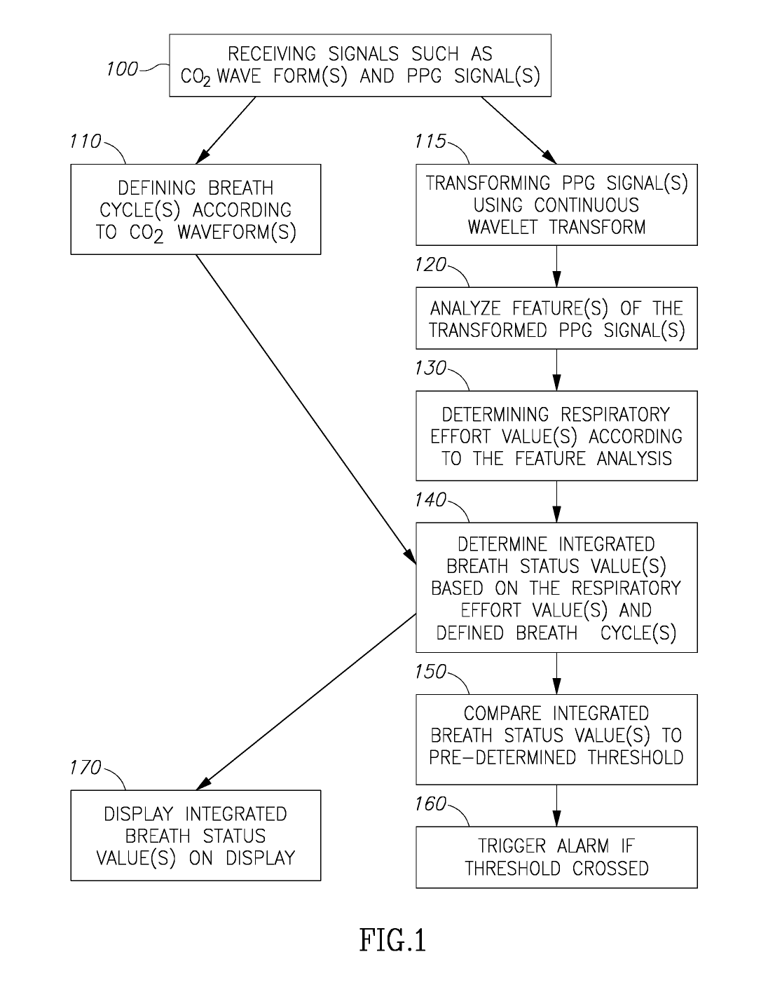

Reference is now made to FIG. 1 which is an illustrative flowchart depicting the steps used to determine an integrated breath status value in accordance with some embodiments.

In step 100, one or more signals may be received, including, for example, a PPG signal and a CO₂ waveform.

Step 110, comprises defining a breath cycle based on the CO₂ waveform.

Step 115 comprises transforming the received PPG signal(s) using a wavelet transform such as a continuous wavelet transform. Once the signal is transformed into a suitable domain, it may be depicted by a suitable representation such as a scalogram. According to some embodiments, step 115 may include filtering the PPG signal(s), mathematically manipulating the PPG signal, convolving the PPG signal with a reference signal, or any combination thereof.

Once the PPG signal(s) is transformed and represented by a suitable representation, one or more features may be analyzed within the scalogram as shown in step 120. For example, parameters such as an energy parameter, morphology, pattern within a region of the scalogram may be calculated. For example, a pulse band or a respiration band may be determined.

In step 130, effort information may be determined based at least in part on the analysis of the features in step 120. As described above, effort may be correlated with changes or the appearance of the features found and analyzed in step 120. For example, breathing effort may be correlated with a change or weighted change in amplitude, energy, amplitude modulation, frequency modulation, and/or scale modulation in the breathing and/or pulse bands. The correlation between effort and the analyzed features may be used to determine quantitative or qualitative values associated with effort. The determined values may, for example, indicate effort or a change of effort.

In step 140, an integrated breath status value may be determined based on the defined breath cycle and the effort value derived from the transformed PPG signal(s). As described above, the integrated breath status value may for example be an effort per breath value.

In step 150 the integrated breath status value of step 140 is compared to a predetermined threshold value. Hence it is determined whether the integrated breath status value has risen above or fallen below the threshold value. If the integrated breath status value crosses the predetermined threshold value, an alert may be issued in step 160. In some embodiments, the alert may be triggered if effort rises above or falls below a threshold value by a particular percentage change, absolute value change, or if the determined effort value crosses the threshold value. Optionally, in step 170 the integrated breath status value or trends thereof may be displayed on a display. A graphical display may be displayed in one, two, or more dimensions and may be fixed or change with time. The graphical representation may be further enhanced by changes in color, pattern, or any other visual representation.

The analysis performed in step 140 may be used in connection with evaluation of respiratory conditions such as for example asthma. Increased effort may be used to detect and/or distinguish between respiratory conditions of different severity. For example, the method may be used to differentiate between mild, moderate, severe and acute severe asthma attacks. In an embodiment, the integrated breath status value may be used in combination with additional parameters typically used to evaluate respiratory conditions. It is understood by the skilled in the art that the above method may be implemented using any human-readable or machine-readable instructions on any suitable system or apparatus, such as those described herein.

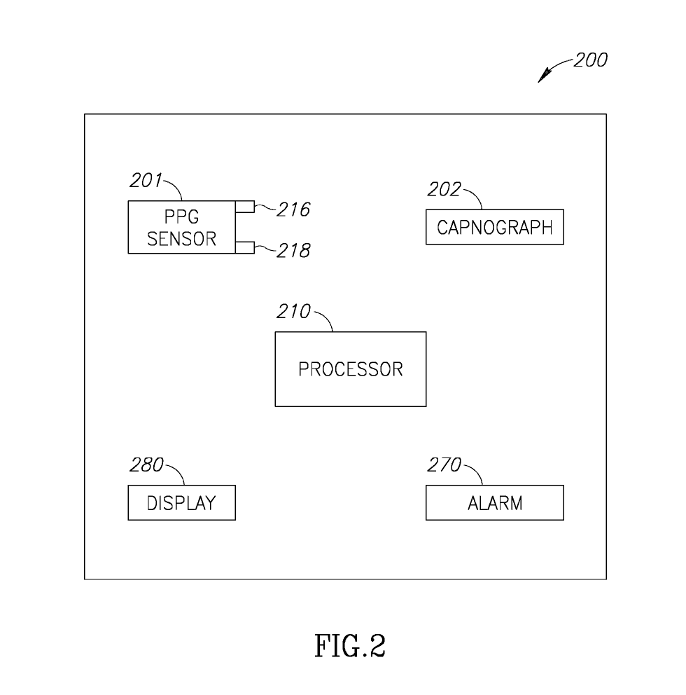

Reference is now made to FIG. 2 which is a perspective view of an exemplary system 200 as described herein. In an embodiment, system 200 includes sensors such as a PPG sensor 201 and a capnograph 202. PPG sensor 201 may include an emitter 216 for emitting light at two or more wavelengths into a patient's tissue. A detector 218 may also be provided in PPG sensor 201 for detecting the light originally from emitter 216 that emanates from the patient's tissue after passing through the tissue.

According to some embodiments, emitter 216 and detector 218 may be on opposite sides of a digit such as a finger or toe, in which case the light that is emanating from the tissue has passed completely through the digit. According to some embodiments, emitter 216 and detector 218 may be arranged so that light from emitter 216 penetrates the tissue and is reflected by the tissue into detector 218, such as a sensor designed to obtain pulse oximetry data from a patient's forehead.

According to some embodiments, system 200 may include a plurality of sensors forming a sensor array in lieu of PPG sensor 201 and capnograph 202 (not shown).

System 200 further includes a processor 210 configured to: define individual breath cycles based on the CO₂ waveforms; generate a transformed signal based on the PPG signals; calculate a respiratory effort value based on the transformed PPG signal; and calculate an integrated breath status value based on the defined breath cycle and the calculated respiratory effort value.

According to some embodiments, PPG sensor 201 and capnograph 202 may be connected to and draw their power from processor 210. According to some embodiments, PPG sensor 201 and capnograph 202 may be wirelessly connected to processor 210. Processor 210 may be configured to calculate the integrated breath status value based at least in part on data received from PPG sensor 201 and capnograph 202.

Optionally, system 200 further includes a display, such as display 280 configured to display the integrated breath status value and/or the trend calculated/obtained from the processor.

According to some embodiments, system 200 may also include an alarm 270 configured to provide an audible sound in the event that the integrated breath status value is not within a predefined normal range.

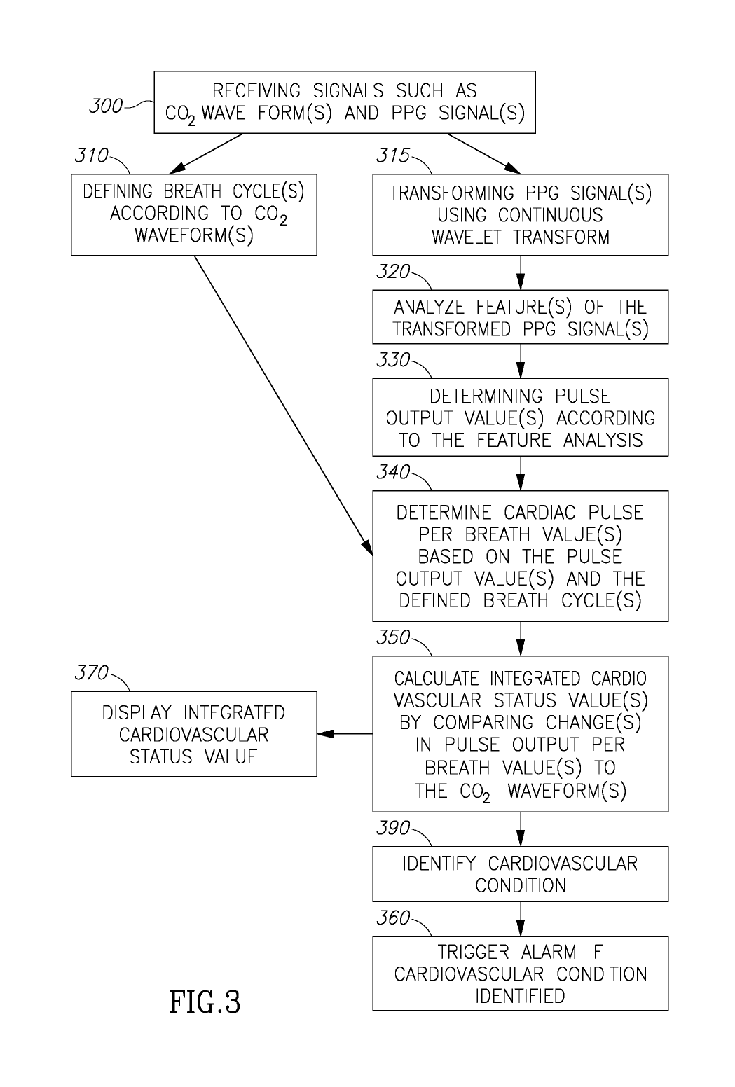

Reference is now made to FIG. 3 which is an illustrative flowchart depicting the steps used to determine an integrated cardiovascular status value in accordance with some embodiments.

In step 300, one or more signals may be received, including, for example, a PPG signal(s) and CO₂ waveform(s).

Step 310, comprises defining a breath cycle based on the CO₂ waveform.

Step 315 comprises generating a pulse output value by transforming the received PPG signal(s) using a wavelet transform such as a continuous wavelet transform. Once the PPG signal(s) is transformed into a suitable domain, it may be depicted by a suitable representation, such as a scalogram. According to some embodiments, step 315 may optionally include filtering the PPG signal, mathematically manipulating the PPG signal, convolving PPG signal with a reference signal, or any combination thereof.

Once the PPG signal(s) is transformed and represented by a suitable representation, one or more features may be analyzed within the scalogram as shown in step 320. For example, parameters such as an energy parameter, morphology, pattern within a region of the scalogram may be calculated. For example, a pulse band or a respiration band may be determined.

In step 330, a pulse output value may be determined based on the analysis performed in step 320.

In step 340, a pulse output per breath value may be determined based on the defined breath cycle and the pulse output value. For example, a pulse rhythm abnormality may be identified based on a detected change in the pulse output per breath value. As described above, pulse rhythm abnormalities may enable identification of cardiovascular irregularities.

In step 350 an integrated cardiovascular status value is determined by comparing a change in the pulse output per breath value to a change in the CO₂ waveform.

Optionally, in step 390 a cardiovascular condition may be identified based on the integrated cardiovascular status value. Furthermore, if a cardiovascular condition is identified, an alert may optionally be issued in step 360. Additionally or alternatively, in step 370 the integrated cardiovascular status value may be displayed on a display. A graphical display may be displayed in one, two, or more dimensions and may be fixed or change with time. The graphical representation may be further enhanced by changes in color, pattern, or any other visual representation. According to some embodiments, respiratory rate, pulse rate or any other suitable physiological information may be displayed together with the integrated breath status value.

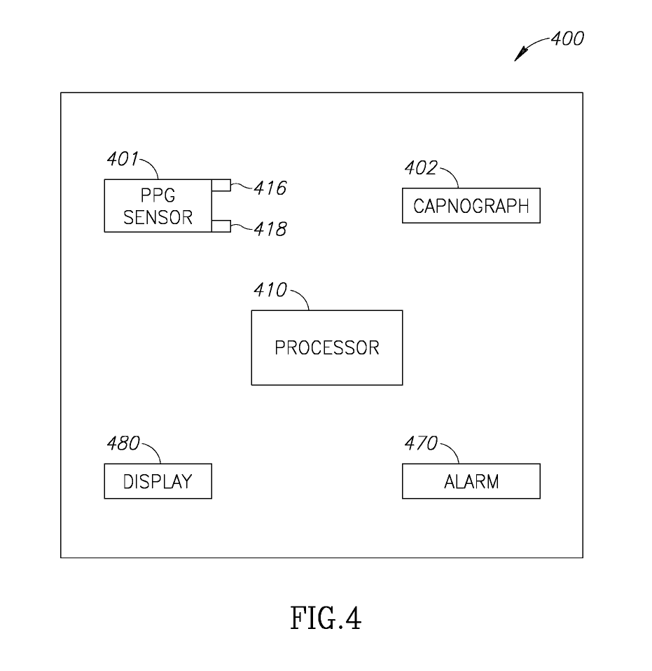

Reference is now made to FIG. 4 which shows a perspective view of an exemplary system 400 used to determine an integrated cardiovascular status value in accordance with some embodiments.

In an embodiment, system 400 includes sensors such as PPG sensor 401 and capnograph 402. PPG sensor 401 may include an emitter 416 for emitting light at two or more wavelengths into a patient's tissue. A detector 418 may also be provided in PPG sensor 401 for detecting the light originally from emitter 416 that emanates from the patient's tissue after passing through the tissue.

According to some embodiments, emitter 416 and detector 418 may be on opposite sides of a digit such as a finger or toe, in which case the light that is emanating from the tissue has passed completely through the digit. According to some embodiments, emitter 416 and detector 418 may be arranged so that light from emitter 416 penetrates the tissue and is reflected by the tissue into detector 418, such as a sensor designed to obtain pulse oximetry data from a patient's forehead.

According to some embodiments, system 400 may include a plurality of sensors forming a sensor array in lieu of PPG sensor 401 and capnograph 402 (not shown).

System 400 further includes a processor 410 configured to: define a breath cycle based on a CO₂ waveform; generate a transformed signal based at least on a PPG signal; identify a pulse output value based on the transformed PPG signal; calculate a pulse output per breath value based at least on the defined breath cycle and the identified pulse output value; and determine the integrated cardiovascular status value based on a comparison of a change in the pulse output per breath and a change in the CO₂ waveform.

According to some embodiments, PPG sensor 401 and capnograph 402 may be connected to and draw their power from processor 410. According to some embodiments, PPG sensor 401 and capnograph 402 may be wirelessly connected to processor 410. Processor 410 may be configured to calculate the integrated breath status value based at least in part on data received from PPG sensor 402 and capnograph 404.

Optionally, system 400 further includes a display, such as display 480 configured to display the integrated cardiovascular status value calculated by and/or obtained from the processor.

According to some embodiments, system 400 may also include an alarm 470 configured to provide an audible sound when a cardiovascular abnormality is identified.

While a number of exemplary aspects and embodiments have been discussed above, those of skill in the art will recognize certain modifications, additions and sub-combinations thereof. It is therefore intended that the following appended claims and claims hereafter introduced be interpreted to include all such modifications, additions and sub-combinations as are within their true spirit and scope.

What is claimed is:

1. A method for calculating an integrated breath status value, the method comprising:
defining a breath cycle based on a CO₂ waveform;
generating a transformed signal based at least on a photoplethysmograph (PPG) signal;
calculating a respiratory effort value based at least on the transformed PPG signal; and
calculating an integrated breath status value based on the defined breath cycle and the calculated respiratory effort value.

2. The method of claim 1, wherein the integrated breath status value is an effort per breath value.

3. The method of claim 1, wherein the integrated breath status value is an weighted effort per breath value further based on at least one additional waveform related parameter, at least one additional PPG related parameter or combinations thereof.

4. The method of claim 3, wherein the at least one additional waveform related parameter is selected from the group consisting of: slope of the CO₂ waveform, slope in a plateau region of the CO₂ waveform, amplitude of the CO₂ waveform, angle of rise, time to rise, run time of the rise, curvature, acceleration, area under the CO₂ waveform, baseline value of the CO₂ waveform and any combination of these parameters.

5. The method of claim 3, wherein the at least one additional PPG related parameter is selected from the group consisting of: respiration rate, heart rate, blood pressure, SpO2, fluid responsiveness, perfusion index and combinations thereof.

6. The method of claim 1, wherein the transformed signal is a continuous wavelet transformed signal and wherein generating the transformed signal comprises generating a scalogram.

7. The method of claim 1, further comprising assessing severity of a respiratory condition of a patient based on the integrated breath status value, wherein the respiratory condition is asthma, chronic obstructive pulmonary disease (COPD), lung injury, pneumonia or combinations thereof.

8. The method of claim 7, wherein assessing the severity of the respiratory condition comprises comparing the integrated breath status value to a predetermined threshold value.

9. The method of claim 8, wherein assessing the severity of the respiratory condition further comprises applying a fuzzy logic, a Bayesian network, a decision tree, a neural network, a radial base function, a linear regression model, a non-linear regression model, an expert system, or any combination thereof.

10. The method of claim 8, further comprising triggering an alarm when the integrated breath status value crosses the predetermined threshold value.

11. The method of claim 7, further comprising identifying an improvement, deterioration or lack of change in the respiratory condition based on a trend in the integrated breath status value.

12. The method of claim 11, further comprising displaying the integrated breath status value and/or the trend on a display.

13. A device for calculating an integrated breath status value, the device comprising a processor configured to:
define a breath cycle based on a CO₂ waveform;
generate a transformed signal based at least on a photoplethysmograph (PPG) signal;
calculate a respiratory effort value based at least on the transformed PPG signal; and
calculate an integrated breath status value based on the defined breath cycle and the calculated respiratory effort value.

14. The device of claim 13, wherein said processor is further configured to assess the severity of a respiratory condition by comparing the integrated breath status value to a predetermined threshold value, wherein the respiratory condition comprise asthma, chronic obstructive pulmonary disease (COPD), lung injury, pneumonia or combinations thereof.

15. The device of claim 14, wherein said processor is further configured to trigger an alarm when the integrated breath status value crosses the predetermined threshold value.

16. The device of claim 14, wherein said processor is further configured to identify an improvement, deterioration or lack of change in the respiratory condition based on a trend in the integrated breath status value.

17. The device of claim 16, wherein said processor is further configured to display the integrated breath status value and/or the trend on a display.

18. A system for calculating an integrated breath status value, the system comprising:
a capnograph configured to provide CO₂ waveforms;
a PPG sensor configured to provide PPG signals; and
a processor configured to:
define a breath cycle based on the CO₂ waveforms;
generate a transformed signal based on the PPG signals;
calculate a respiratory effort value based on the transformed PPG signal; and
calculate an integrated breath status value based on the defined breath cycle and the calculated respiratory effort value.

19. The system of claim 18, further comprising an alarm configured to be triggered when the integrated breath status value crosses a predetermined threshold.

20. The system of claim 18, further comprising a display configured to display the calculated integrated breath status value.

21. A method for calculating an integrated cardiovascular status value, the method comprising:
defining a breath cycle based on a CO₂ waveform;
generating a transformed signal based at least on a PPG signal;
calculating a pulse output value based on the transformed PPG signal;
calculating a pulse output per breath value based at least on the defined breath cycle and the calculated pulse output value; and determining the integrated cardiovascular status value based on a comparison of a change in the pulse output per breath value and a change in the CO₂ waveform.

22. The method of claim 21, wherein the change in the pulse output per breath value may proceed, follow or be concurrent with the change in the CO₂ waveform.

23. The method of claim 21, further comprising identifying a cardiovascular condition based on the integrated cardiovascular status value.

24. The method of claim 23, further comprising identifying an improvement, deterioration or lack of change in the cardiovascular condition.

25. The method of claim 23, wherein the cardiovascular condition is selected from the group consisting of: cardiac arrest, atrial flutter, atrial fibrillation, sinus tachycardia, ventricular tachycardia, ventricular fibrillation, bradycardia, bigeminy, trigeminy, ectopic beats, asystole and any combination thereof.

26. The method of claim 23, further comprising triggering an alarm when a cardiovascular condition is identified.

27. The method of claim 21, further comprising differentiating between cardiovascular conditions and respiratory conditions based on the integrated cardiovascular status value.

28. The method of claim 21, further comprising displaying the integrated cardiovascular status value on a display.

29. The method of claim 21, wherein the transformed signal is a continuous wavelet transformed signal and wherein generating the transformed signal comprises generating a scalogram.

30. The method of claim 21, further comprising calculating a confidence level of the integrated cardiovascular status value.

31. A device for calculating an integrated cardiovascular status value, the device comprising a processor configured to:
define a breath cycle based on a CO₂ waveform;
generate a transformed signal based at least on a PPG signal;
calculate a pulse output value based on the transformed PPG signal;
calculate a pulse output per breath value based at least on the defined breath cycle and the calculated pulse output value; and
determine the integrated cardiovascular status value based on a comparison of a change in the pulse output per breath and a change in the CO₂ waveform.

32. The device of claim 31, wherein said processor is further configured to identify a cardiovascular condition based on the integrated cardiovascular status value.

33. The device of claim 32, wherein said processor is further configured to identify an improvement, deterioration or lack of change in the cardiovascular condition.

34. The device of claim 31, wherein said processor is further configured to display the integrated cardiovascular status value on a display.

35. The device of claim 31, wherein said processor is further configured to trigger an alarm when a cardiovascular condition is identified.

36. A system for calculating an integrated cardiovascular status value, the system comprising:
a capnograph configured to provide CO₂ waveforms;
a PPG sensor configured to provide PPG signals; and
a processor configured to:
define a breath cycle based on the CO₂ waveform;
generate a transformed signal based at least on the PPG signal;
calculate a pulse output value based on the transformed PPG signal;
calculate a pulse output per breath value based at least on the defined breath cycle and the calculated pulse output value; and
determine the integrated cardiovascular status value based on a comparison of a change in the pulse output per breath and a change in the CO₂ waveform.

37. The system of claim 36, further comprising an alarm configured to be triggered when a cardiovascular condition is identified.

38. The system of claim 36, further comprising a display configured to display the integrated cardiovascular status value.
